(19) United States
(12) Patent Application Publication
(10) Pub. No.: US 2013/0137772 A1
(43) Pub. Date: May 30, 2013

(54) HYDROXYPOLYAMINE SALTS

(71) Applicant: Raymond J. Bergeron, Gainesville, FL (US)

(72) Inventor: Raymond J. Bergeron, Gainesville, FL (US)

(21) Appl. No.: 13/306,980

(22) Filed: Nov. 29, 2011

(51) Int. Cl. A61K 31/132; C07C 309/04; A61P 1/18; A61P 35/00; A61P 13/12; A61P 1/16; C07C 211/13; A61K 31/205

(52) U.S. Cl. 514/578; 564/512; 562/114; 514/674

(57) ABSTRACT

A salt of a polyamine having the formula:

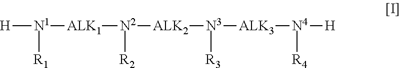

with a pharmaceutically acceptable organic or inorganic acid, wherein at least one of the bridging groups ALK₁, ALK₂ and ALK₃ contains at least one —CH(OH)-group which is not alpha- to any of the nitrogen atoms.

### BACKGROUND OF THE INVENTION

1. Field of the Invention

The invention relates to novel hydroxy substituted polyamines having valuable therapeutic and other biological properties.

2. Discussion of the Prior Art

In recent years, a great deal of attention has been focused on the polyamines, e.g., spermidine, norspermidine, homospermidine, 1,4-diaminobutane (putrescine) and spermine. These studies have been largely directed at the biological properties of the polyamines probably because of the role they play in proliferative processes. It was shown early on that the polyamine levels in dividing cells, e.g., cancer cells, are much higher than in resting cells. See Janne et al, A. Biochim. Biophys. Acta., Vol. 473, page 241 (1978); Fillingame et al, Proc. Natl. Acad. Sci. U.S.A., Vol. 72, page 4042 (1975); Metcalf et al, J. Am. Chem. Soc., Vol. 100, page 2551 (1978); Flink et al, Nature (London), Vol. 253, page 62 (1975); and Pegg et al, Polyamine Metabolism and Function, Am. J. Cell. Physiol., Vol. 243, pages 212-221 (1982).

Several lines of evidence indicate that polyamines, particularly spermidine, are required for cell proliferation: (i) they are found in greater amounts in growing than in non-growing tissues; (ii) prokaryotic and eukaryotic mutants deficient in polyamine biosynthesis are auxotrophic for polyamines; and (iii) inhibitors specific for polyamine biosynthesis also inhibit cell growth. Despite this evidence, the precise biological role of polyamines in cell proliferation is uncertain. It has been suggested that polyamines, by virtue of their charged nature under physiological conditions and their conformational flexibility, might serve to stabilize macromolecules such as nucleic acids by anion neutralization. See Dkystra et al, Science, Vol. 149, page 48 (1965); Russell et al, Polyamines as Biochemical Markers of Normal and Malignant Growth (Raven, New York, 1978); Hirschfield et al, J. Bacteriol., Vol. 101, page 725 (1970); Hafner et al, J. Biol. Chem., Vol. 254, page 12419 (1979); Cohn et al, J. Bacteriol., Vol. 134, page 208 (1978); Pohjatipelto et al, Nature (London), Vol. 293, page 475 (1981); Mamont et al, Biochem. Biophys. Res. Commun., Vol. 81, page 58 (1978); Bloomfield et al, Polyamines in Biology and Medicine (D. R. Morris and L. J. Morton, eds., Dekker, New York, 1981), pages 183-205; Gosule et al, Nature, Vol. 259, page 333 (1976); Gabbay et al, Ann. N.Y. Acad. Sci., Vol. 171, page 810 (1970); Suwalsky et al, J. Mol. Biol., Vol. 42, page 363 (1969); and Liguori et al, J. Mol. Biol., Vol. 24, page 113 (1968).

However, regardless of the reason for increased polyamine levels, the phenomenon can be and has been exploited in chemotherapy. See Sjoerdsma et al, Butterworths Int. Med. Rev.: Clin. Pharmacol. Thera., Vol. 35, page 287 (1984); Israel et al, J. Med. Chem., Vol. 16, page 1 (1973); Morris et al, Polyamines in Biology and Medicine, Dekker, New York, page 223 (1981); and Wang et al, Biochem. Biophys. Res. Commun., Vol. 94, page 85 (1980).

Because of the role the natural polyamines play in proliferation, a great deal of effort has been invested in the development of polyamine analogues as anti-proliferatives [Cancer Res., Vol. 49, “The role of methylene backbone in the anti-proliferative activity of polyamine analogues on L1210 cells,” Bergeron et al, pages 2959-2964 (1989); J. Med. Chem., Vol. 31, “Synthetic polyamine analogues as antineoplastics,” Bergeron et al, pages 1183-1190 (1988); Polyamines in Biochemical and Clinical Research, “Regulation of polyamine biosynthetic activity by spermidine and spermine—a novel antiproliferative strategy,” Porter et al, pages 677-690 (1988); Cancer Res., Vol. 49, “Major increases in spermidine/spermine-N¹-acetyl transferase activity by spermine analogues and their relationship to polyamine depletion and growth inhibition in L1210 cells,” Basu et al, pages 6226-6231 (1989); Biochem. J., Vol. 267, “Induction of spermidine/spermine N¹-acetyltransferase activity in Chinese-hamster ovary cells by N¹,N¹¹-bis(ethyl)norspermine and related compounds,” Pegg et al, pages 331-338 (1990); Biochem. J., Vol. 268, “Combined regulation of ornithine and S-adenosylmethionine decarboxylases by spermine and the spermine analogue N¹N¹²-bis(ethyl)spermine,” Porter et al, pages 207-212 (1990); Cancer Res., Vol. 50, “Effect of N¹,N¹⁴-bis(ethyl)-homospermine on the growth of U-87 MG and SF-126 on human brain tumor cells,” Basu et al, pages 3137-3140 (1990); and Biochem. Biophys. Res. Commun., Vol. 152, “The effect of structural changes in a polyamine backbone on its DNA binding properties,” Stewart, pages 1441-1446 (1988)]. These efforts have included the design of new synthetic methods [J. Org. Chem., Vol. 45, “Synthesis of N⁴-acylated N¹,N⁸-bis(acyl)spermidines: An approach to the synthesis of siderophores,” Bergeron et al, pages 1589-1592 (1980); Synthesis, “Reagents for the selective acylation of spermidine, homospermidine and bis-[3-aminopropyl]amine,” Bergeron et al, pages 732-733 (1981); Synthesis, “Reagents for the selective secondary functionalization of linear triamines,” Bergeron et al, pages 689-692 (1982); Synthesis, “Amines and polyamines from nitriles,” Bergeron et al, pages 782-785 (1984); J. Org. Chem., Vol. 49, “Reagents for the stepwise functionalization of spermidine, homospermidine and bis-[3-aminopropyl]amine,” Bergeron et al, page 2997 (1984); Accts. Chem. Res., Vol. 19, “Methods for the selective modification of spermidine and its homologues,” Bergeron, pages 105-113 (1986); Bioorg. Chem., Vol. 14, “Hexahydropyrimidines as masked spermidine vectors in drug delivery,” Bergeron et al, pages 345-355 (1986); J. Org. Chem., Vol. 53, “Reagents for the stepwise functionalization of spermine,” Bergeron et al, pages 3108-3111 (1988); J. Org. Chem., Vol. 52, “Total synthesis of (.+−.)-15-Deoxyspergualin,” Bergeron et al, pages 1700-1703 (1987); J. Org. Chem., Vol. 56, “The total synthesis of Alcaligin,” Bergeron et al, pages 586-593 (1991); and CRC Handbook on Microbial Iron Chelates, “Synthesis of catecholamide and hydroxamate siderophores,” Bergeron et al, pages 271-307 (1991)] for the production of these analogues, as well as extensive biochemical studies focused on the mechanism by which these compounds act [Cancer Res., Vol. 46, “A comparison and characterization of growth inhibition by .alpha.-Difluoromethylornithine (DFMO), and inhibitor of ornithine decarboxylase and N¹,N⁸-bis(ethyl)spermidine (BES), an apparent regulator of the enzyme,” Porter et al, pages 6279-6285 (1986); Cancer Res., Vol. 47, “Relative abilities of bis(ethyl) derivatives of putrescine, spermidine and spermine to regulate polyamine biosynthesis and inhibit L1210 leukemia cell growth,” Porter et al, pages 2821-2825 (1987); Cancer Res., Vol. 49, “Correlation between the effects of polyamine analogues on DNA conformation and cell growth,” Basu et al, pages 5591-5597 (1989); Cancer Res., Vol. 49, “Differential response to treatment with the bis(ethyl)polyamine analogues between human small cell lung carcinoma and undifferentiated large cell lung carcinoma in culture,” Casero et al, pages 639-643 (1988); Mol. Pharm., Vol. 39, “Selective cellular depletion of mitochondrial DNA by the polyamine analog, N¹,N¹²-bis(ethyl)spermine, and its relationship to polyamine structure and function,” Vertino et al, pages 487-494 (1991); Biochem. and Biophys. Res. Comm., Vol. 157, “Modulation of polyamine biosynthesis and transport by oncogene transfection,” Chang et al, pages 264-270 (1988); and Biopolymers, Vol. 26, “Structural determinants of spermidine-DNA interactions,” Vertino et al, pages 691-703 (1987)]. The mechanistic investigations have encompassed uptake studies, impact on polyamine analogues on polyamine pools and polyamine biosynthetic enzymes, as well as their effects on translational and transcriptional events.

Anti-neoplastic analogues of the naturally occurring polyamines, pharmaceutical compositions and methods of treatment are also disclosed in the following pending patent application Ser. No. 08/231,692 filed Apr. 25, 1994, as well as in U.S. Pat. Nos. 5,091,576 issued Feb. 25, 1992; 5,128,353 issued Jul. 7, 1992; and 5,173,505 issued Dec. 22, 1992. The disclosures of each of the foregoing applications and patents are incorporated herein by reference.

Many of the biologically and pharmacologically valuable polyamines, however, present troublesome metabolic properties in that they are metabolized after administration to the whole animal to potentially toxic metabolites, several of which have a protracted half-life in animals.

Diethylnorspermine (DENSPM) and its metabolites are found in all of the tissues of mice treated with the drug, with the liver and kidney having the highest level of metabolites. These catabolic products included N¹-ethylnorspermine (MENSPM), N¹-ethylnorspermidine (MENSPD), N¹-ethyl-1,3-diaminopropane (MEDAP) and norspermidine (NSPD), suggesting that DENSPM is metabolized by (1) N-deethylation and (2) stepwise removal of 3-aminopropyl equivalents by spermine/spermidine N¹-acetyl transferase (SSAT)/polyamine oxidase (PAO).

Diethylhomospermine is an example of a polyamine recently found to have potent activity as an anti-neoplastic and anti-diarrheal agent. Its metabolic profile indicates the highest concentration of the polyamine and its principal metabolites, N¹-ethylhomospermine (MEHSPM) and homospermine (HSPM), in the liver and kidney. N-deethylation is a key metabolic step in processing DEHSPM; however, HSPM does not undergo further metabolism. The accumulation and persistence of HSPM in the tissues of DEHSPM-treated animals is especially striking. Even three weeks after a seven day schedule of DEHSPM, 35% of the drug administered to mice remains in the liver and kidney as drug or metabolites. Interestingly, 90% of the drug remaining in the animal at this time is in the form of HSPM. It is quite clear that the increased chronic toxicity of DEHSPM over N¹,N⁴-diethylnorspermine (DENSPM) is related to the buildup of HSPM.

The key to a less toxic DEHSPM-like therapeutic agent is one in which the metabolites can be quickly cleared from the tissues. Again, because of the aminobutyl fragments of HSPM, this metabolite cannot be processed through the polyamine biosynthetic network; thus, it remains in the tissues for protracted periods of time. Neither the primary nor the secondary nitrogens of HSPM offer an opportunity for facile conversion to an easily cleared metabolite. Certainly, the methylene backbones cannot be easily oxidized to an excretable metabolite.

In U.S. Pat. No. 5,962,533 there are described novel derivatives of therapeutically and biologically active polyamines which are metabolized to products quickly and easily cleared from animal tissues. These derivatives are polyamines having the formula:

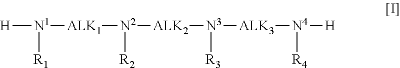

or its possible stereoisomers, derivatives, prodrugs or complexes wherein:
a] R₁ and R₄ may be the same or different and are alkyl, aryl, aryl alkyl or cycloalkyl, optionally having an alkyl chain interrupted by at least one etheric oxygen atom;
R₂ and R₃ may be the same or different and are R₁, R₄ or H;
N₁, N₂, N₃ and N₄ are nitrogen atoms capable of protonation at physiological pH's;
ALK₁, ALK₂ AND ALK₃ may be the same or different and are straight or branched chain alkylene bridging groups having 1 to 4 carbon atoms which effectively maintain the distance between the nitrogen atoms such that the polyamine:
(i) is capable of uptake by a target cell upon administration of the polyamine to a human or non-human animal or is capable of binding to at least one polyamine site of a receptor located within or on the surface of a cell upon administration of the polyamine to a human or non-human animal; and
(ii) upon uptake by the target cell, competitively binds via an electrostatic interaction between the positively charged nitrogen atoms to biological counter-anions;
the polyamine, upon binding to the biological counter-anion in the cell, functions in a manner biologically different than the intracellular polyamines; and,
b] at least one of said bridging groups ALK₁, ALK₂ and ALK₃ contains at least one —CH(OH)— group which is not alpha- to any of the nitrogen atoms.

In U.S. Pat. No. 6,160,022, there is described a method for chemically resecting the pancreas of a human or non-human animal by treatment thereof with a hydroxypolyamine described in U.S. Pat. No. 5,962,533.

It is an object of the present invention to provide novel derivatives of therapeutically and biologically active polyamines.

It is another object of the invention to provide novel pharmaceutical compositions and methods of treating human and non-human animals with the novel polyamine derivatives.

It is another object of the invention to provide novel pharmaceutical compositions and methods for modifying the compositions of biological materials.

### SUMMARY OF THE INVENTION

The above and other objects are realized by the present invention, one embodiment of which comprises a salt of a polyamine having the formula:

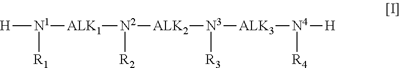

with a pharmaceutically acceptable organic acid; or its possible stereoisomers, derivatives, prodrugs or complexes wherein:
a] R₁ and R₄ may be the same or different and are alkyl, aryl, aryl alkyl or cycloalkyl, optionally having an alkyl chain interrupted by at least one etheric oxygen atom;
R₂ and R₃ may be the same or different and are R₁, R₄ or H;
N₁, N₂, N₃ and N₄ are nitrogen atoms capable of protonation at physiological pH's;
ALK₁, ALK₂ AND ALK₃ may be the same or different and are straight or branched chain alkylene bridging groups having 1 to 4 carbon atoms which effectively maintain the distance between the nitrogen atoms such that the polyamine:
(i) is capable of uptake by a target cell upon administration of the polyamine to a human or non-human animal or is capable of binding to at least one polyamine site of a receptor located within or on the surface of a cell upon administration of the polyamine to a human or non-human animal; and
(ii) upon uptake by the target cell, competitively binds via an electrostatic interaction between the positively charged nitrogen atoms to biological counter-anions;
the polyamine, upon binding to the biological counter-anion in the cell, functions in a manner biologically different than the intracellular polyamines; and,
b] at least one of said bridging groups ALK₁, ALK₂ and ALK₃ contains at least one —CH(OH)— group which is not alpha- to any of the nitrogen atoms.

A second embodiment of the invention relates to pharmaceutical composition comprising (1) a pharmaceutically effective amount of a polyamine salt of formula [1], its possible stereoisomers, derivatives, prodrugs or complexes, or (2) a salt of the polyamine of formula [I] with a pharmaceutically acceptable inorganic acid, its possible stereoisomers, derivatives, prodrugs or complexes and a pharmaceutically acceptable carrier therefore

A further embodiment of the invention concerns a method of treating a human or non-human animal in need thereof comprising administering thereto a pharmaceutically effective amount of one of the above-described polyamine salts.

A still further embodiment of the invention comprises compositions for the isolation of islet of Langerhans cells from acinar cells in a material containing both cells comprising solutions, suspensions or mixtures of an inorganic or organic acid salt as described above in a pharmaceutically acceptable carrier having a concentration of the salt sufficient to destroy the acinar cells but insufficient to deleteriously affect the islets of Langerhans cells

An additional embodiment of the invention concerns methods for the isolation of islet of Langerhans cells from acinar cells in a material containing both types of cells comprising treating the material with a solution, suspension or mixture of a of an inorganic or organic acid salt as described above in a pharmaceutically acceptable carrier for a time sufficient to digest the acinar cells but insufficient to deleteriously affect the islets of Langerhans cells.

### DETAILED DESCRIPTION OF THE INVENTION

The present invention is predicated on the discovery that the biological properties of the polyamine salts of the invention depends on the nature of the acid employed to form the salt. Thus, the take-up and metabolization of the above described polyamine salts depends upon whether the acid employed to form the salt is an organic or inorganic acid.

The tissue distribution and metabolism of DEHSPM, (HO)₂DEHSPM HCl Salt and (HO)₂DEHSPM Mesylate Salt in various organs were evaluated utilizing the following protocol:

### Tissue Distribution and Metabolism of DEHSPM, (HO)₂DEHSPM HCl Salt and (HO)₂DEHSPM Mesylate Salt

1) DEHSPM, (HO)₂DEHSPM HCl salt, and (HO)₂DEHSPM mesylate salt were dissolved in sterile normal saline; the pH was not adjusted.
2) Female CD-1 mice were given the drugs at equimolar dosages intraperitoneally once daily for 5 days.
3) The animals, n=3-4 per group, were euthanized 4, 8, or 24 h post-last dose.
4) The kidney, liver, and pancreas were removed and assessed for their polyamine content via HPLC analysis.
5) The polyamine concentration data (mean±STD) are expressed as nmol/g wet weight of tissue. SPM, spermine; SPD, spermidine; PUT, putrescine; N.D., not determined.
6) A one-tailed t test assuming unequal variance was performed on the tissue polyamine concentration data. A value of p<0.05 was considered significant.

TABLE 1
Control

| | SPM | SPD | PUT |
|---|---|---|---|
| Kidney | | | |
| 4 h | 624 ± 43 | 389 ± 53 | 18 ± 16 |
| 8 h | 579 ± 3 | 291 ± 40 | 27 ± 10 |
| 24 h | 597 ± 34 | 319 ± 14 | 28 ± 1 |
| Liver | | | |
| 4 h | 719 ± 59 | 1003 ± | 0 ± 0 |
| | | 132 | |
| 8 h | 687 ± 26 | 896 ± 65 | 0 ± 0 |
| 24 h | 741 ± 56 | 1133 ± | 14 ± 17 |
| | | 96 | |
| Pancreas | | | |
| 4 h | 1368 ± | 6249 ± | 49 ± 25 |
| | 43 | 63 | |
| 8 h | 1233 ± | 6657 ± | 58 ± 5 |
| | 120 | 201 | |
| 24 h | 1201 ± | 6528 ± | 43 ± 5 |
| | 177 | 320 | |

TABLE 2
DEHSPM 18.6 mg/kg/d IP x 5 d

| | SPM | SPD | PUT | DEHSPM | MEHSPM | HSPM |
|---|---|---|---|---|---|---|
| Kidney | | | | | | |
| 4 h | 243 ± 49 | 270 ± 61 | 0 ± 0 | 428 ± 95 | 37 ± 7 | 799 ± |
| | | | | | | 188 |
| 8 h | 240 ± 52 | 159 ± 4 | 0 ± 0 | 425 ± 82 | 44 ± 7 | 971 ± |
| | | | | | | 262 |
| 24 h | 223 ± 61 | 150 ± 37 | 0 ± 0 | 260 ± 19 | 5 ± 10 | 838 ± |
| | | | | | | 143 |
| Liver | | | | | | |
| 4 h | 764 ± 49 | 540 ± 77 | 0 ± 0 | 148 ± 46 | 7 ± 11 | 432 ± 44 |
| 8 h | 899 ± 40 | 710 ± | 0 ± 0 | 144 ± 7 | 16 ± 14 | 542 ± 50 |
| | | 132 | | | | |
| 24 h | 809 ± 45 | 578 ± | 21 ± 15 | 79 ± 10 | 0 ± 0 | 550 ± 65 |
| | | 150 | | | | |
| Pancreas | | | | | | |
| 4 h | 1000 ± | 6054 ± | 43 ± 10 | 330 ± 37 | 0 ± 0 | 65 ± 10 |
| | 124 | 878 | | | | |
| 8 h | 1033 ± | 5789 ± | 66 ± 33 | 339 ± 59 | 9 ± 16 | 71 ± 22 |
| | 290 | 339 | | | | |
| 24 h | 1146 ± | 5807 ± | 62 ± 29 | 284 ± 50 | 0 ± 0 | 75 ± 21 |
| | 107 | 262 | | | | |

TABLE 3
(HO)₂DEHSPM HCl Salt 20 mg/kg/d IP x 5 d

| | | | | (HO)₂DEHSPM | | |
|---|---|---|---|---|---|---|
| | SPM | SPD | PUT | 4HCl Salt | (HO)₂MEHSPM | (HO)₂HSPM |
| Kidney | | | | | | |
| 4 h | 544 ± | 327 ± 86 | 31 ± 27 | 852 ± 202 | N.D. | 50 ± 19 |
| | 14 | | | | | |
| 8 h | 641 ± | 275 ± 16 | 29 ± 5 | 871 ± 85 | N.D. | 70 ± 6 |
| | 33 | | | | | |
| 24 h | 588 ± | 304 ± 30 | 28 ± 6 | 618 ± 82 | N.D. | 76 ± 7 |
| | 5 | | | | | |
| Liver | | | | | | |
| 4 h | 875 ± | 1050 ± 9 | 0 ± 0 | 248 ± 17 | N.D. | 70 ± 5 |
| | 128 | | | | | |
| 8 h | 1071 ± | 1175 ± 190 | 20 ± 17 | 228 ± 55 | N.D. | 103 ± 14 |
| | 7 | | | | | |
| 24 h | 837 ± | 884 ± 156 | 7 ± 14 | 136 ±50 | N.D. | 102 ± 13 |
| | 13 | | | | | |
| Pancreas | | | | | | |
| 4 h | 1153 ± | 5868 ± 505 | 32 ± 3 | 217 ± 27 | N.D. | 56 ± 11 |
| | 159 | | | | | |
| 8 h | 1476 ± | 6343 ± 596 | 50 ± 7 | 223 ± 54 | N.D. | 60 ± 9 |
| | 101 | | | | | |
| 24 h | 1177 ± | 6386 ± 309 | 36 ± 5 | 209 ± 37 | N.D. | 58 ± 14 |
| | 161 | | | | | |

TABLE 4
(HO)₂DEHSPM Mesylate Salt 30.3 mg/kg/d IP x 5 d

| | | | | (HO)₂DEHSPM | | |
|---|---|---|---|---|---|---|
| | SPM | SPD | PUT | Mesylate | (HO)₂MEHSPM | (HO)₂HSPM |
| Kidney | | | | | | |
| 4 h | 680 ± | 404 ± 24 | 7 ± 12 | 541 ± 65 | N.D. | 77 ± 6 |
| | 15 | | | | | |
| 8 h | 655 ± | 265 ± 5 | 0 ± 0 | 533 ± 84 | N.D. | 89 ± 14 |
| | 41 | | | | | |
| 24 h | 601 ± | 239 ± 9 | 26 ± 5 | 360 ± 43 | N.D. | 97 ± 5 |
| | 29 | | | | | |
| Liver | | | | | | |
| 4 h | 908 ± | 1044 ± 177 | 0 ± 0 | 168 ± 32 | N.D. | 79 ± 7 |
| | 55 | | | | | |
| 8 h | 987 ± | 1108 ± 146 | 0 ± 0 | 135 ± 3 | N.D. | 116 ± 15 |
| | 49 | | | | | |
| 24 h | 807 ± | 910 ±10 | 0 ± 0 | 73 ± 11 | N.D. | 88 ± 5 |
| | 138 | | | | | |
| Pancreas | | | | | | |
| 4 h | 1108 ± | 5717 ± 426 | 36 ± 10 | 131 ± 27 | N.D. | 50 ± 6 |
| | 15 | | | | | |
| 8 h | 1333 ± | 6460 ± 280 | 57 ± 15 | 153 ± 11 | N.D. | 70 ± 2 |
| | 103 | | | | | |

TABLE 5
Control

| Kidney | SPM | SPD | PUT |
|---|---|---|---|
| 4 h | 624 ± 43 | 389 ± 53 | 18 ± 16 |
| 8 h | 579 ± 3 | 291 ± 40 | 27 ± 10 |
| 24 h | 597 ± 34 | 319 ± 14 | 28 ± 1 |

DEHSPM 18.6 mg/kg/d IP x 5 d

| Kidney | SPM | SPD | PUT | DEHSPM | MEHSPM | HSPM |
|---|---|---|---|---|---|---|
| 4 h | 243 ± 49 | 270 ± 61 | 0 ± 0 | 428 ± 95 | 37 ± 7 | 799 ± 188 |
| | | | | | | |
| 8 h | 240 ± 52 | 159 ± 4 | 0 ± 0 | 425 ± 82 | 44 ± 7 | 971 ± 262 |
| | | | | | | |
| 24 h | 223 ± 61 | 150 ± 37 | 0 ± 0 | 260 ± 19 | 5 ± 10 | 838 ± 143 |
| | | | | | | |

(HO)₂DEHSPM•4HCl Salt 20 mg/kg/d IP x 5 d

| | | | | (HO)₂DEHSPM | | |
|---|---|---|---|---|---|---|
| Kidney | SPM | SPD | PUT | 4HCl Salt | (HO)₂MEHSPM | (HO)₂HSPM |
| 4 h | 544 ± 14 | 327 ± 86 | 31 ± 27 | 852 ± 202 | N.D. | 50 ± 19 |
| | | | | | | |
| 8 h | 641 ± 33 | 275 ± 16 | 29 ± 5 | 871 ± 85 | N.D. | 70 ± 6 |
| | ▴ | | | | | |
| 24 h | 588 ± 5 | 304 ± 30 | 28 ± 6 | 618 ± 82 | N.D. | 76 ± 7 |

(HO)₂DEHSPM Mesylate Salt 30.3 mg/kg/d IP x 5 d

| | | | | (HO)₂DEHSPM | | |
|---|---|---|---|---|---|---|
| Kidney | SPM | SPD | PUT | Mesylate | (HO)₂MEHSPM | (HO)₂HSPM |
| 4 h | 680 ± 15 | 404 ± 24 | 7 ± 12 | 541 ± 65 | N.D. | 77 ± 6 |
| | | | | ▪ | | □ |
| 8 h | 655 ± 41 | 265 ± 5 | 0 ± 0 | 533 ± 84 | N.D. | 89 ± 14 |
| | ▴ | | ▪ | ▪ | | |
| 24 h | 601 ± 29 | 239 ± 9 | 26 ± 5 | 360 ± 43 | N.D. | 97 ± 5 |
| | | ▪ | | ▪ | | □ |

 < Control
▴ > Control
▪ < (HO)₂DEHSPM•4HCl
□ > (HO)₂DEHSPM HCl

TABLE 6

| Liver | SPM | SPD | PUT |
|---|---|---|---|
| 4 h | 719 ± 59 | 1003 ± 132 | 0 ± 0 |
| 8 h | 687 ± 26 | 896 ± 65 | 0 ± 0 |
| 24 h | 741 ± 56 | 1133 ± 96 | 14 ± 17 |

DEHSPM 18.6 mg/kg/d IP x 5 d

| Liver | SPM | SPD | PUT | DEHSPM | MEHSPM | HSPM |
|---|---|---|---|---|---|---|
| 4 h | 764 ± 49 | 540 ± 77 | 0 ± 0 | 148 ± 46 | 7 ± 11 | 432 ± 44 |
| | | | | | | |
| 8 h | 899 ± 40 | 710 ± 132 | 0 ± 0 | 144 ± 7 | 16 ± 14 | 542 ± 50 |
| | ▴ | | | | | |
| 24 h | 809 ± 45 | 578 ± 150 | 21 ±15 | 79 ± 10 | 0 ± 0 | 550 ± 65 |
| | | | | | | |

(HO)₂DEHSPM•4HCl Salt 20 mg/kg/d IP x 5 d

| | | | | (HO)₂DEHSPM | | |
|---|---|---|---|---|---|---|
| Liver | SPM | SPD | PUT | HCl Salt | (HO)₂MEHSPM | (HO)₂HSPM |
| 4 h | 875 ± 128 | 1050 ± 9 | 0 ± 0 | 248 ± 17 | N.D. | 70 ± 5 |
| 8 h | 1071 ± 7 | 1175 ± 190 | 20 ± 17 | 228 ± 55 | N.D. | 103 ± 14 |
| | ▴ | | | | | |
| 24 h | 837 ± 13 | 884 ± 156 | 7 ± 14 | 136 ± 50 | N.D. | 102 ± 13 |
| | ▴ | | | | | |

(HO)₂DEHSPM Mesylate Salt 30.3 mg/kg/d IP x 5 d

| Liver | SPM | SPD | PUT | (HO)₂DEHSPM | (HO)₂MEHSPM | (HO)₂HSPM |
|---|---|---|---|---|---|---|
| 4 h | 908 ± 55 | 1044 ± 177 | 0 ± 0 | 168 ± 32 | N.D. | 79 ± 7 |
| | ▴ | | | ▪ | | |
| 8 h | 987 ± 49 | 1108 ± 146 | 0 ± 0 | 135 ± 3 | N.D. | 116 ± 15 |
| | ▴▪ | | | ▪ | | |
| 24 h | 807 ± 138 | 910 ± 10 | 0 ± 0 | 73 ± 11 | N.D. | 88 ± 5 |
| | | | | ▪ | | |

 < Control
▴ > Control
▪ < (HO)₂DEHSPM•4HCl Salt

TABLE 7
Control

| Pancreas | SPM | SPD | PUT |
|---|---|---|---|
| 4 h | 1368 ± 43 | 6249 ± 63 | 49 ± 25 |
| 8 h | 1233 ± 120 | 6657 ± 201 | 58 ± 5 |
| 24 h | 1201 ± 177 | 6528 ± 320 | 43 ± 5 |

DEHSPM 18.6 mg/kg/d IP x 5 d

| Pancreas | SPM | SPD | PUT | DEHSPM | MEHSPM | HSPM |
|---|---|---|---|---|---|---|
| 4 h | 1000 ± 124 | 6054 ± 878 | 43 ± 10 | 330 ± 37 | 0 ± 0 | 65 ± 10 |
| | | | | | | |
| 8 h | 1033 ± 290 | 5789 ± 339 | 66 ± 33 | 339 ± 59 | 9 ± 16 | 71 ± 22 |
| | | | | | | |
| 24 h | 1146 ± 107 | 5807 ± 262 | 62 ± 29 | 284 ± 50 | 0 ± 0 | 75 ± 21 |
| | | | | | | |

(HO)₂DEHSPM HCl Salt 20 mg/kg/d IP x 5 d

| | | | | (HO)₂DEHSPM | | |
|---|---|---|---|---|---|---|
| Pancreas | SPM | SPD | PUT | 4HCl Salt | (HO)₂MEHSPM | (HO)₂HSPM |
| 4 h | 1153 ± 159 | 5868 ± 505 | 32 ± 3 | 217 ± 27 | N.D. | 56 ± 11 |
| 8 h | 1476 ± 101 | 6343 ± 596 | 50 ± 7 | 223 ± 54 | N.D. | 60 ± 9 |
| | ▴ | | | | | |
| 24 h | 1177 ± 161 | 6386 ± 309 | 36 ± 5 | 209 ± 37 | N.D. | 58 ± 14 |
| | | | | | | |

(HO)₂DEHSPM Mesylate Salt 30.3 mg/kg/d IP x 5 d

| | | | | (HO)₂DEHSPM | | |
|---|---|---|---|---|---|---|
| Pancreas | SPM | SPD | PUT | Mesylate | (HO)₂MEHSPM | (HO)₂HSPM |
| 4 h | 1108 ± 15 | 5717 ± 426 | 36 ± 10 | 131 ± 27 | N.D. | 50 ± 6 |
| | | | | ▪ | | |
| 8 h | 1333 ± 103 | 6460 ± 280 | 57 ± 15 | 153 ± 11 | N.D. | 70 ± 2 |
| 24 h | 1273 ± 210 | 6147 ± 223 | 31 ± 3 | 134 ± 14 | N.D. | 75 ± 4 |
| | | | | ▪ | | □ |

 < Control
▴ > Control
▪ < (HO)₂DEHSPM•4HCl
□ > (HO)₂DEHSPM HCl

The above results demonstrate, unexpectedly, that organic acid salts of the hydroxypolyamines are the taken-up and metabolized to a far less extent than the inorganic acid salts. The two types of acid salts were administered in vivo and it was determined that the inorganic acid salts were absorbed and metabolized by the kidneys, livers and pancreases in amounts 1.64, 1.67 and 1.55 times, respectively, that of organic acid salts.

Thus, in instances where it is desired to concentrate greater amounts of the hydroxypolyamines in certain organs, it has been unexpectedly found that the utilization of inorganic acid salts yields much better results than organic acid salts. Correspondingly, where it is desirable to treat a patient with a compromised internal organ such that lesser amounts of the hydroxypolyamine are delivered thereto, it has been unexpectedly been found that the administration of organic acid salts results in the absorption and metabolization of smaller amounts thereof; however, said smaller amounts are still effective to achieve the desired therapeutic result.

Moreover, it has unexpectedly been found that the salts of the invention are valuable for destroying the acinar cells in pancreases, in vitro, while preserving the islet of Langerhans cells thereof. The pancreas is a gland organ in the digestive and endocrine system of vertebrates. It is both an endocrine gland producing several important hormones, including insulin, glucagon, and somatostatin, as well as a digestive organ, secreting pancreatic juice containing digestive enzymes that assist the absorption of nutrients and the digestion in the small intestine. These enzymes help to further break down the carbohydrates, proteins, and lipids in the chyme.

Under a microscope, stained sections of the pancreas reveal two different types of parenchymal tissue. Lightly staining clusters of cells are called islets of Langerhans, which produce hormones that underlie the endocrine functions of the pancreas. Darker staining cells form acini connected to ducts. Acinar cells belong to the exocrine pancreas and secrete digestive enzymes into the gut via a system of ducts. The pancreas is a dual-function gland, having features of both endocrine and exocrine glands.

The part of the pancreas with endocrine function is made up of approximately a million cell clusters called islets of Langerhans. The islets are a compact collection of endocrine cells arranged in clusters and cords and are crisscrossed by a dense network of capillaries. The capillaries of the islets are lined by layers of endocrine cells in direct contact with vessels, and most endocrine cells are in direct contact with blood vessels, by either cytoplasmic processes or by direct apposition. According to the volume The Body, by Alan E. Nourse, the islets are “busily manufacturing their hormone and generally disregarding the pancreatic cells all around them, as though they were located in some completely different part of the body.” The islet of Langerhans plays an imperative role in glucose metabolism and regulation of blood glucose concentration.

The pancreas as an exocrine gland helps out the digestive system. It secretes pancreatic juice that contains digestive enzymes that pass to the small intestine. These enzymes help to further break down the carbohydrates, proteins, and lipids (fats) in the chyme. In humans, the secretory activity of the pancreas is regulated directly via the effect of hormones in the blood on the islets of Langerhans and indirectly through the effect of the autonomic nervous system on the blood flow.

Isolation and purification of a specific cell population is an important issue in many areas of cell biology. Several methods of purification of cells have been used over the years, including centrifugal separation based upon size or density, cloning and immunological (antibody) recognition and separation, among others.

Over the years, many different compounds have been used to form density gradients to enable particles to be separated according to their size and/or buoyant density. Some common materials for this purpose include Ficoll®, Percoll®, cesium chloride and dextran. While these materials have been used for the separation of cells, they have certain drawbacks (outlined below) which make them not particularly well-suited for this task.

When cells are separated using centrifugation, they may be separated by size or buoyant density, or to a minor extent, by charge or other related external surface characteristic. With respect to separation processes relying upon differences in size, when particles in solution are subjected to a centrifugal field, they move in the direction of the force applied, and in general, larger particles will move faster than smaller particles. Therefore, when cells differ greatly in size, a reasonable purification may be obtained by low speed centrifugation in a suitable medium.

In cell suspensions containing a mixed population of cells, cells of similar sizes may have different buoyant densities, whereas cells of different sizes may have the same densities. Cells having different buoyancy characteristics may be separated on density gradients, such as continuous gradients or discrete step (discontinuous) gradients. In either case, the principle is that the cells will migrate through the gradient medium until they reach a point where the density of the medium equals the density of the cells, in the case of a continuous gradient, or where the cells are sandwiched at the interface in between a medium having a lower density and a medium having a greater density than the cells, in the case of a discontinuous gradient. This method results in the cells being disposed in discrete bands within between the media.

An example of the need for rapid isolation and purification of cells is the desirability of acquiring large quantities of pure insulin producing cells from a pancreas for purposes of transplanting them into a diabetic patient. The pancreas is the organ responsible for insulin production. Specifically, insulin is produced and regulated by areas of the pancreas known as the Islets of Langerhans, referred to herein as islet cells or islets, which are the endocrine cells of the pancreas. Such cells comprise a small percentage of the pancreas (around 2%). The major cellular component of the pancreas consists of exocrine tissue including acinar and ductal cells, and it has been shown to be a formidable task to purify the islet cells from the acinar cells. Gray and Morris, Transplantation (1987) 43:321.

Islet cell isolation and purification is currently being performed by density gradient separation based upon the principle of density differences between the isolated islets and the acinar cells. Generally the dispersed pancreatic preparation is placed in a discontinuous density gradient solution containing Ficoll®, Percoll® or dextran, all well-known density gradient materials. There are several disadvantages to prior art methods of islet cell isolation and preparation utilizing such materials. For one, the method is cumbersome, time consuming and labor intensive since several gradients are required to be layered and the islets must be carefully removed from within these multiple gradients. Another disadvantage is that the method yields inconsistent results since the density of the islet cells and acinar cells may change during the process as a result of edema in the cells caused by the materials used in the separation. Another serious disadvantage of the prior art methods is that the cells are subjected for substantial periods of time to the gradient material which may be toxic, and is at least detrimental to the viability of the cells. This is particularly the case because the currently used gradient solutions such as Ficoll®, Percoll® and dextran are not physiological solutions thus causing both osmotic and ionic stresses on the cells. Ficoll® is known to be toxic to cells as well as mutagenic. Percoll® also causes cellular damage. Dextran in the concentrations used in gradient separation may cause cellular damage as a result of the osmotic stresses applied to the cells. Furthermore, none of the gradient separation materials are capable of preserving cells for any substantial periods of time.

The salts of the invention may be prepared according to the methods disclosed in U.S. Pat. No. 5,962,533.

It will thus be understood that all of the possible diastereoisomers of the hydroxy polyamines of the above structural formula will be effective active agents in the compositions and methods of the invention. Accordingly, as utilized when describing the present invention, the above structural formula includes all of the diastereoisomers, as well as racemates, of the hydroxy polyamine salts embraced thereby.

For the utilities mentioned herein, the amount required of active agent, the frequency and the mode of its administration and/or application will vary with the identity of the agent concerned and with the nature and severity of the condition being treated and is, of course, ultimately at the discretion of the responsible physician or veterinarian. In general, however, a suitable dose of agent for all of the above-described conditions will lie in the range of about 0.01 mg/kg to about 30 mg/kg, and preferably about 0.5 mg/kg to about 10 mg/kg, of mammal body weight being treated. The composition is preferably administered parenterally (intravenously, intradermally, intraperitoneally, intramuscularly or subcutaneously), but may also be administered orally for a period of time sufficient to result in the resection of the exocrine portion of the pancreas. The precise period of time will depend in each case, of course, upon the animal under treatment and the dosage employed. By monitoring the function of the animal according to conventional methods during administration of the hydroxy polyamine salts, the time of treatment required can be accurately gauged.

While it is possible for the agents to be administered as the raw substances, it is preferable to present them as a pharmaceutical formulation. The formulations of the present invention, both for veterinary and human use, comprise the agents together with one or more pharmaceutically acceptable carriers therefore and, optionally, other therapeutic ingredients. The carrier(s) must be “acceptable” in the sense of being compatible with the other ingredients of the formulation and not deleterious to the recipient thereof. Desirably, the formulations should not include oxidizing agents and other substances with which the agents are known to be incompatible. The formulations may conveniently be presented in unit dosage form and may be prepared by any of the methods well known in the art of pharmacy. All methods include the step of bringing into association the agent with the carrier which constitutes one or more accessory ingredients. In general, the formulations are prepared by uniformly and intimately bringing into association the agent with the carrier(s) and then, if necessary, dividing the product into unit dosages thereof.

Formulations suitable for oral administration may be in the form of discrete units such as capsules, cachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion.

A tablet may be made by compressing or molding the active ingredient optionally with one or more accessory ingredients. Compressed tablets may be prepared by compressing, in a suitable machine, the active ingredient in a free-flowing form such as a powder or granules, optionally mixed with a binder, lubricant, inert diluent, surface active agent or dispensing agent. Molded tablets may be made by molding, in a suitable machine, a mixture of the powdered active ingredient and a suitable carrier moistened with an inert liquid diluent.

Formulations suitable for parenteral administration conveniently comprise sterile aqueous preparations of the agents which are preferably isotonic with the blood of the recipient. Suitable such carrier solutions include phosphate buffered saline, saline, water, lactated ringers or dextrose (5% in water). Such formulations may be conveniently prepared by admixing the agent with water to produce a solution or suspension which is filled into a sterile container and sealed against bacterial contamination. Preferably, sterile materials are used under aseptic manufacturing conditions to avoid the need for terminal sterilization.

Formulations for oral or parenteral administration may optionally contain one or more additional ingredients among which may be mentioned preservatives such as methyl hydroxybenzoate, chlorocresol, metacresol, phenol and benzalkonium chloride. Such materials are of special value when the formulations are presented in multi-dose containers.

Buffers may also be included to provide a suitable pH value for the formulation and suitable materials include sodium phosphate and acetate. Sodium chloride or other appropriate salts may be used to render a formulation isotonic with the blood. If desired, the formulation may be filled into the containers under an inert atmosphere such as nitrogen or may contain an anti-oxidant and are conveniently presented in unit dose or multi-dose form, for example, in a sealed ampoule.

It will be appreciated that while the agents described herein form acid addition salts and carboxylic acid salts, the biological activity thereof will reside in the agent itself. These salts may be used in human and in veterinary medicine and presented as pharmaceutical formulations in the manner and in the amounts (calculated as the base) described hereinabove, and it is then preferable that the acid moiety be pharmacologically and pharmaceutically acceptable to the recipient.

The active agent or pharmaceutically acceptable derivatives or salts thereof may also be mixed with other pharmaceutically active materials that do not interfere with the desired action or with materials that enhance or supplement the desired action. Examples of appropriate other agents include antibiotics, anti-fungals, anti-virals, antihistamines, immunosuppressants and other anti-inflammatory or analgesic compounds the like.

The entire disclosures and contents of all literature and patent references cited herein are incorporated in their entirety by reference.

I claim:

1. A salt of a polyamine having the formula: 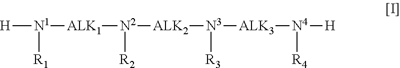
with a pharmaceutically acceptable organic acid; or its possible stereoisomers, derivatives, prodrugs or complexes wherein:
a] R₁ and R₄ may be the same or different and are alkyl, aryl, aryl alkyl or cycloalkyl, optionally having an alkyl chain interrupted by at least one etheric oxygen atom;
R₂ and R₃ may be the same or different and are R₁, R₄ or H;
N₁, N₂, N₃ and N₄ are nitrogen atoms capable of protonation at physiological pH's;
ALK₁, ALK₂ AND ALK₃ may be the same or different and are straight or branched chain alkylene bridging groups having 1 to 4 carbon atoms which effectively maintain the distance between the nitrogen atoms such that the polyamine:
(i) is capable of uptake by a target cell upon administration of the polyamine to a human or non-human animal or is capable of binding to at least one polyamine site of a receptor located within or on the surface of a cell upon administration of the polyamine to a human or non-human animal; and
(ii) upon uptake by the target cell, competitively binds via an electrostatic interaction between the positively charged nitrogen atoms to biological counter-anions;
the polyamine, upon binding to the biological counter-anion in the cell, functions in a manner biologically different than the intracellular polyamines; and,
b] at least one of said bridging groups ALK₁, ALK₂ and ALK₃ contains at least one —CH(OH)— group which is not alpha- to any of the nitrogen atoms.

2. The polyamine salt of claim 1 wherein said polyamine has the formula:

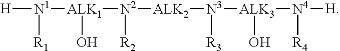

3. The polyamine salt of claim 1 wherein said polyamine has the formula:

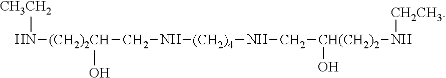

4. The polyamine salt of claim 1, wherein said organic acid is selected from the group consisting of 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, isobutyric acid, lactic acid (DL), lactobionic acid, lauric acid, maleic acid, malic acid (−L), malonic acid, mandelic acid (DL), methanesulfonic acid, naphthalene-1,5-disulfonic acid, naphthalene-2-sulfonic acid, nicotinic acid, oleic acid, oxalic acid, palmitic acid, pamoic acid, proprionic acid, pyroglutamic acid (−L), salicylic acid, sebacic acid, stearic acid, succinic acid, tartaric acid (+L), thiocyanic acid, toluenesulfonic acid (p), undecylenic acid.

5. The polyamine salt of claim 1 wherein said organic acid is methanesulfonic acid.

6. A pharmaceutical composition comprising (1) a pharmaceutically effective amount of a polyamine salt of claim 1, its possible stereoisomers, derivatives, prodrugs or complexes, (2) a salt of the polyamine of formula [I] with a pharmaceutically acceptable inorganic acid, its possible stereoisomers, derivatives, prodrugs or complexes or (3) a mixture thereof and a pharmaceutically acceptable carrier therefore.

7. The pharmaceutical composition of claim 6 wherein said inorganic acid is hydrochloric acid, hydrobromic acid, sulfuric acid, nitric acid or phosphoric acid.

8. The pharmaceutical composition of claim 6 wherein said polyamine salt is of an acid that maximizes the tissue distribution of said polyamine salt, stereoisomer, derivative, prodrug, complex or metabolite thereof in a target organ.

9. The pharmaceutical composition of claim 8 wherein said target organ is the kidney, liver or pancreas and the acid is an inorganic acid.

10. The pharmaceutical composition of claim 9 wherein said acid is hydrochloric acid.

11. The pharmaceutical composition of claim 6 wherein said polyamine salt is of an acid that minimizes the tissue distribution of said polyamine salt, stereoisomer, derivative, prodrug, complex or metabolite thereof in a target organ.

12. The pharmaceutical composition of claim 11 wherein said target organ is the kidney, liver or pancreas and the acid is an organic acid.

13. The pharmaceutical composition of claim 12 wherein said acid is methane sulfonic acid.

14. The pharmaceutical composition of claim 6 wherein the amount of polyamine salt is pharmaceutically effective for the treatment of pancreatic cancer.

15. A method of treating a human or non-human animal in need thereof comprising administering thereto a pharmaceutically effective amount of a polyamine salt of claim 1.

16. A method according to claim 15 wherein said polyamine salt is of an acid that minimizes the tissue distribution of said polyamine salt, stereoisomer, derivative, prodrug, complex or metabolite thereof in a target organ.

17. The method of claim 16 wherein said target organ is the kidney, liver or pancreas and the acid is an organic acid.

18. The method of claim 17 wherein said acid is methane sulfonic acid.

19. The method of claim 17 wherein said target organ is the pancreas and said animal is in need of treatment for pancreatic cancer.

20. A method according to claim 15 wherein said polyamine salt is of an acid that maximizes the tissue distribution of said polyamine salt, stereoisomer, derivative, prodrug, complex or metabolite thereof in a target organ.

21. A method according to claim 20 wherein said target organ is the kidney, liver or pancreas and the acid is an inorganic acid.

22. The method of claim 20 wherein said acid is hydrochloric acid.

23. The method of claim 15 wherein the amount of polyamine salt is pharmaceutically effective for the treatment of pancreatic cancer.

24. A composition for the isolation of islet of Langerhans cells from acinar cells in a material containing both comprising a solution, suspension or mixture of a salt of claim 1 in a pharmaceutically acceptable carrier having a concentration of said salt sufficient to destroy said acinar cells but insufficient to deleteriously affect said islets of Langerhans cells

25. The composition of claim 24 wherein said material is pancreatic.

26. A method for the isolation of islet of Langerhans cells from acinar cells in a material containing both comprising treating said material with a solution, suspension or mixture of a salt of claim 1 in a pharmaceutically acceptable carrier for a time sufficient to digest said acinar cells but insufficient to deleteriously affect said islets of Langerhans cells

27. The method of claim 26 wherein said material is pancreatic.
